(19) United States
(12) Patent Application Publication
(10) Pub. No.: US 2021/0052682 A1
(43) Pub. Date: Feb. 25, 2021

(54) Herbal Supplement for Liver Purification

(71) Applicant: Wang Nan Yip, Forest Hills, NY (US)

(72) Inventor: Wang Nan Yip, Forest Hills, NY (US)

(21) Appl. No.: 16/547,842

(22) Filed: Aug. 22, 2019

(51) Int. Cl. A61K 36/258; A61K 36/28; A61K 36/15; A61K 36/725; A61K 36/734; A61K 31/717; A61K 47/42; A23L 33/00; A61P 1/16

(52) U.S. Cl. CPC A61K 36/258; A61K 36/28; A61K 36/15; A61K 36/725; A61P 1/16; A61K 31/717; A61K 47/42; A23L 33/30; A61K 36/734

(57) ABSTRACT

Purification of the liver and removal of toxins from the liver may be improved through the ingestion of an herbal supplement containing a specific composition of materials. The herbal supplement contains an effective amount of Echinacea extract, pin pollen extract, royal jelly extract, Crataegus pinnatifida extract, Ziziphus jujuba Mill. extract, and Ginseng extract. The pin pollen extract contains a specific mixture of four flavonoids. The mixture of four flavonoids contains 37.21 percent of a first flavonoid, 22.61 percent of a second flavonoid, 14.94 percent of a third flavonoid, and 25.24 percent of a fourth flavonoid.

### FIELD OF THE INVENTION

The present invention pertains generally to nutritional supplements and more particularly to a nutritional supplement composition having synergistically active minerals and constituents suited to reduce hepatotoxic substances and increase hepatic functionality.

### BACKGROUND OF INVENTION

Medicinal herbs and substances are well known and have been used for centuries. There is a desire among individuals to utilize natural components to improve the health of users. The liver is an organ in the body which functions to metabolize and break down toxic substances found in the body. These metabolized components can cause dysfunction to the liver. The ability of the liver to metabolize harmful substances can be diminished over time with the continued presence of toxic substances in the liver.

Scientific studies have shown that combinations of medicinal herbs have favorably influenced the course of acute hepatic injury induced in rats by the oral intake of acetaminophen and/or ethyl alcohol. Combinations of medicinal herbs have decreased hepatic toxicity in rats. Combinations of medicinal herbs have significantly ameliorated acetaminophen-induced toxicity, as well as overcoming acetaminophen-induced hepatotoxicity in rats simultaneously challenged with ethyl alcohol ingestion. Combinations of medicinal herbs have caused decrease in elevations of liver enzymes aspartate (particularly aminotransferase and alanine aminotransferase) which were used as biomarkers of hepatotoxicity.

What is needed is a substance for ingestion which prevents or lessens the dysfunction of the liver caused by toxicity. While synthetic drugs and substances may be utilized, consumers prefer to have an all-natural substance which can be utilized for the same purpose and same result. The present invention is thus directed to a combination of medicinal herbs which can be utilized to reduce or prevent mild to moderate hepatic injury.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The invention is directed toward an herbal supplement to be utilized for liver purification. The composition of the supplement comprises: 1170-1430 mg Echinacea extract; 720-880 mg pine pollen extract; 540-660 mg royal jelly extract; 540-660 mg Crataegus pinnatifida extract; 450-550 mg Ziziphus jujuba Mill. extract; and 198-242 mg Ginseng extract.

The composition of the supplement may be: Echinacea extract in the amount of 1200-1400 mg; pine pollen extract in the amount of 760-840 mg; royal jelly extract in the amount of 570-630 mg; Crataegus pinnatifida extract in the amount of 570-630 mg; Ziziphus jujuba Mill. extract in the amount of 475-525 mg; and Ginseng extract in the amount of 209-231 mg.

The composition of the supplement may be: Echinacea extract in the amount of 1250-1350 mg; pine pollen extract in the amount of 780-820 mg; royal jelly extract in the amount of 590-130 mg; Crataegus pinnatifida extract in the amount of 590-610 mg; Ziziphus jujuba Mill. extract in the amount of 490-510 mg; and Ginseng extract in the amount of 215-225 mg.

The composition of the supplement may be: Echinacea extract in the amount of 1300 mg; pine pollen extract in the amount of 800 mg; royal jelly extract in the amount of 600 mg; Crataegus pinnatifida extract in the amount of 600 mg; Ziziphus jujuba Mill. extract in the amount of 500 mg; and Ginseng extract in the amount of 220 mg.

The pine pollen extract may comprise a mixture of flavonoids. The mixture of flavonoids comprises a first flavonoid, a second flavonoid, a third flavonoid, and a fourth flavonoid.

When the pine pollen extract is subjected to gas chromatography the first flavonoid has a retention time of 3.885 minutes; the second flavonoid has a retention time of 5.554 minutes; the third flavonoid has a retention time of 6.023 minutes; and the fourth flavonoid has a retention time of 6.740 minutes.

The mixture of flavonoids may comprise 37.21 percent of the first flavonoid; 22.61 percent of the second flavonoid; 14.94 percent of the third flavonoid; and 25.24 percent of the fourth flavonoid.

The liver purification supplement may further comprise gelatine. The liver purification supplement may further comprise vegetable magnesium stearate. The liver purification supplement may further comprise microcrystalline cellulose. The liver purification supplement may further comprise vegetable magnesium stearate.

Still other embodiments of the present invention will become readily apparent to those skilled in this art from the following description wherein there is shown and described the embodiments of this invention, simply by way of illustration of the best modes suited to carry out the invention. As it will be realized, the invention is capable of other different embodiments and its several details are capable of modifications in various obvious aspects all without departing from the scope of the invention. Accordingly, the drawing and descriptions will be regarded as illustrative in nature and not as restrictive.

### BRIEF DESCRIPTION OF THE DRAWINGS

Various exemplary embodiments of this invention will be described in detail, wherein like reference numerals refer to identical or similar components, with reference to the following figures, wherein:

FIG. 1 is graph of calibration of gas chromatography equipment; and

FIG. 2 is a graph of gas chromatography of pine pollen extract.

### DETAILED DESCRIPTION OF THE PREFERRED EMBODIMENT

The claimed subject matter is now described with reference to the drawings. In the following description, for purposes of explanation, numerous specific details are set forth in order to provide a thorough understanding of the claimed subject matter. It may be evident, however, that the claimed subject matter may be practiced with or without any combination of these specific details, without departing from the spirit and scope of this invention and the claims.

The herbal supplement compound comprises a mixture of Echinacea extract, pine pollen extract, royal jelly extract, Crataegus pinnatifida extract, Ziziphys jujuba Mill extract, and Ginseng extract. The separate components are treated and mixed together to create the beneficial herbal compound. First, the materials are pretreated to remove impurities. The impurities are removed from Crataegus pinnatifida, Ziziphys jujuba Mill, and Ginseng. The Crataegus pinnatifida, Ziziphys jujuba Mill (nucleated), and Ginseng are cut into pieces. The pieces, along with Echinacea, are soaked in a cleaning solution for approximately fifteen minutes to remove any heavy metals and pesticide residue. The cleaning solution is preferably 5% concentration (created by mixing 5 kg concentrated liquid with 100 kg of water. After soaking in the cleaning solution, the materials are then rinsed twice with clean water and dried. The cleaned components are then collected.

The cleaned materials are then placed in 85% ethanol for a period of substantially 24 hours. The cleaned materials are placed in a percolating tank and 85% ethanol is added to the tank. Preferably, the ethanol is placed in the tank until it covers the cleaned materials. In the preferred embodiment the ethanol is higher than the cleaned materials in the tank by about five to ten centimeters. The tank is then percolated at a rate of approximately 120 to 140 ml per minute. If needed, the level of liquid can be maintained by adding in additional 85% ethanol to the tank.

The initial percolated liquid is collected. The amount of collected liquid may be any amount but preferably the amount of collected liquid is 1.5 times the amount of decoction. The initial percolated liquid is stored. The percolation within the tank is continued and the percolated liquid is collected, either at a continuous rate or at a differential rate. The percolated liquid is collected until the color of the collected liquid is lightened (or until an amount of percolated liquid weighing approximately 6 to 7 times the weight or mass of initial cleansed materials.

The collected percolated liquid is then concentrated into a paste. The concentration is performed preferably through a means of evaporation of the 85% ethanol under reduced pressure. The temperature during concentration should not exceed 80° C. The ethanol may then be condensed and recycled for further use. The paste created preferably has a density of 1.08 g/ml although other densities of the paste may be utilized. The paste is then ground mechanically or manually into a powder. This process ends in an initial composition paste.

The Ziziphys jujuba Mill (nucleated) may be prepared separately from the initial composition and then added in. In this method, the Ziziphus jujuba Mill (nucleated) is soaked for two hours in filtered alkaline water. The filtered alkaline water should preferably be between 7.5° C. and 70° C. The amount of filtered alkaline water utilized should weigh nine times the weight of Ziziphus jujuba Mill although other amounts of filtered alkaline water may be utilized. The mixture is then filtered and the dregs of Ziziphus jujuba Mill are collected. The dregs of Ziziphus jujuba Mill are then soaked in water for one hour. The amount of water may be any amount although preferably amount of water used is seven times the weight of the Ziziphus jujuba Mill (i.e. 7 kg of water for every 1 kg of Ziziphus jujuba Mill). The mixture is filtered again and the dregs of Ziziphus jujuba Mill are collected. The dregs of Ziziphus jujuba Mill are then combined with the dregs from the pretreatment and are boiled in water for 30 minutes. Any amount of water may be used but preferably the water weighs seven times the weight of the combined dregs (i.e. 7 kg of water for every 1 kg of dregs). The boiled mixture is then filtered. The filtrate is concentrated into the initial composition paste having a density of 1.08 g/ml although other densities may be realized. The paste is then ground into a powder mechanically or manually. This secondary process ends in a Ziziphus jujuba Mill paste.

Silica gel is then mixed with the initial composition paste and the Ziziphus jujuba Mill paste. First the silica gel is heated to 100° C. for 2 hours. The silica gel is then mixed with the initial composition paste and the Ziziphus jujuba Mill paste. This mixture is dried into a powder. The drying may be accomplished by heating the mixture or reducing the air pressure over the mixture. In the preferred embodiment the mixture is dried by a spray drying process. In this process, the air inlet temperate should be approximately 165° C.-170° C. and the outlet air temperature should be approximately 85° C.-90° C. Once dried into a powder, the total powder is divided. Two-thirds of the total powder is dry granulated. The resulting granules are then mixed with remaining one-third of the total powder. In the final steps the mixture is then combined with pine pollen extract and royal jelly powder. The final composition is placed into empty gelatin capsules for consumption.

The production of the product should be conducted under hygenic conditions. All equipment and appliances should be sanitized and in hygenic condition. Personnel who operate the method should wear protective clothing and otherwise take measures to prevent contamination of the final product. The final product must pass the microbial test.

The process of spray drying is performed by machinery configured to produce a dry powder by rapidly drying a material with hot gas. This method is preferred for creating consistent particle size. Any type of hot gas or hot air may be used in the drying process. The process include spraying the material to be dried through an atomizer or spray nozzle. The drop sizes may be any size during the spraying process. In the preferred embodiment the end product is a free flowing and loose powder. The spray dryer may dry the compound in a single stage or in multiple stages. The powder may be recycled through the drying process in continuous flow. The hot gas may be passed over the compound as co-current or counter-current.

The process of dry granulation is used to form granules of the compound without the use of a liquid solution. This process may be completed by compacting the powder under high pressure with a swaying granulator or a roll compactor. The result of the dry granulation process may be a series of larger tablets or a continuous sheet of compacted material. The tablets or sheet may then be milled to a desired particle size.

The composition may also include trace amount of microcrystalline cellulose and vegetable magnesium stearate.

The Echinacea utilized is any herbaceous flowering plant from the Genus Echinacea. Any part of the Echinacea plant may be utilized—such as the flower, the leaves, or the root.

Crataegus pinnatifida is a plant also known as mountain hawthorn which is a tree bearing fruit. The fruit of the Crataegus pinnatifida is utilized in the composition of the supplement. The fruit may be utilized in any form, such as fresh fruit or dried fruit.

The ginseng utilized is a root of a plant from the genus Panax. Any species of the genus Panax may be utilized.

The Ziziphus jujube Mill. is a fruit belonging to the Rhamnaceous family of plants. The Ziziphus jujube Mill. contains biologically active ingredients, including Vitamin C, phenolics, flavonoids, triterpenic acids, and polysaccharides. Phytochemical studies of jujube fruits have shown the fruits to have biological effects such as anticancer, anti-inflammatory, antiobesity, immunostimulating, antioxidant, hepatoprotective, and gastrointestinal protective activities and inhibition of foam cell formation in macrophages. Any Ziziphus jujube Mill. fruit may be utilized to create the compound.

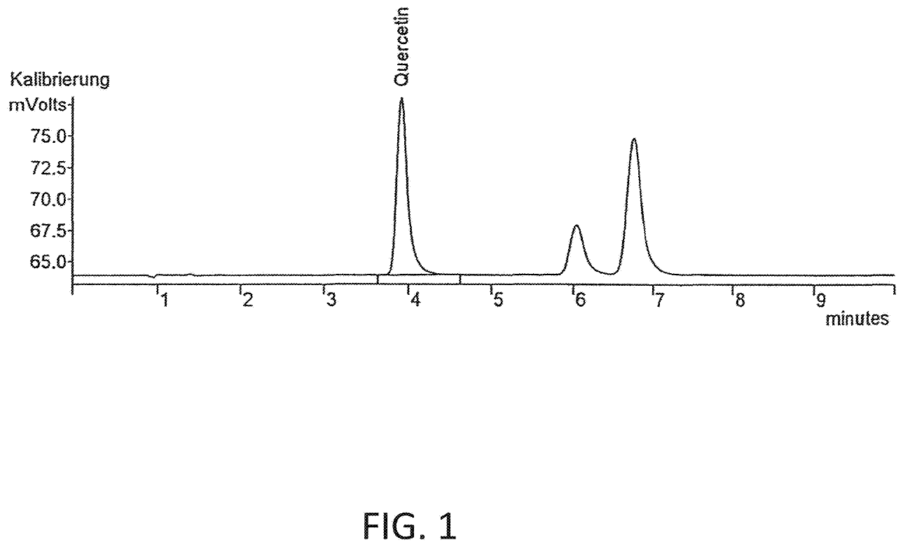

The pollen extract utilized is preferably pine pollen extract. The primary active ingredients in the pine pollen extract are flavonoids. Gas chromatography of the pine pollen extract identifies different flavonoids present which have an effect in the inventive composition. The following gas chromatography charts illustrate the preferred embodiment of the pine pollen extract utilized. Table 1 illustrates the gas chromatography equipment calibration through usage of the plant flavonoid quercetin. The gas chromatography equipment calibration result is illustrated in FIG. 1.

TABLE 1

| Peak | Ret. Time | Peak | | Area | Sep. | Width ½ |
|---|---|---|---|---|---|---|
| No | (min) | Name | Result | (counts) | Code | (sec) |
| 1 | 3.913 | Quercetin | 100.00 | 132294 | BB | 8.0 |
| | | Totals | 100.00 | 132294 | | |

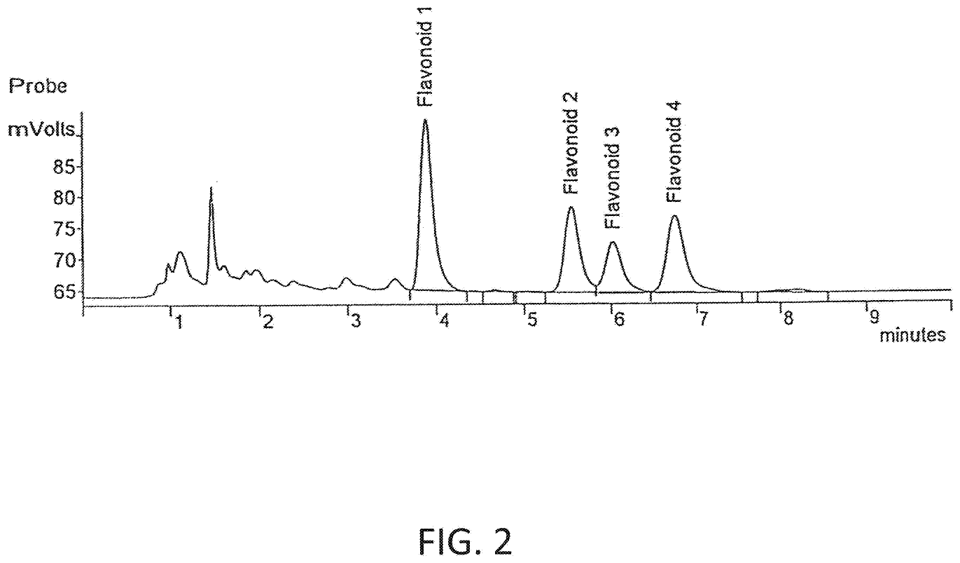

The gas chromatography results of the pine pollen extract used in the compound is illustrated in FIG. 2. These results are set forth below in Table 2.

TABLE 2

| Peak | Ret. Time | Peak | Area | | Sep. | Width ½ |
|---|---|---|---|---|---|---|
| No | (min) | Name | (counts) | Result | Code | (sec) |
| 1 | 3.885 | Flavonoid 1 | 278513 | 37.21 | BB | 8.9 |
| 2 | 5.554 | Flavonoid 2 | 169236 | 22.61 | PV | 11.1 |
| 3 | 6.023 | Flavonoid 3 | 111810 | 14.94 | VV | 11.9 |
| 4 | 6.740 | Flavonoid 4 | 188895 | 25.24 | VB | 12.8 |
| | | Totals | 748454 | 100.00 | | |

The royal jelly extract utilized is standard royal jelly harvested from any bee colony. Royal jelly is collected from individual queen cells from the honeycomb when queen larvae are only a few days old. Royal jelly is primarily 67% water, 12.5% protein, 11% simple sugars (monosaccharides), 6% fatty acids and 3.5% 10-hydroxy-2-decenoic acid (10-HDA).

The final product may be any size and contain any amount of the combined mixture. In the preferred embodiment the preferred composition is set forth below in Table 3:

TABLE 3

| Ingredient | Amount | |
|---|---|---|
| Echinacea Extract | 1300 | mg |
| Pine Pollen Extract | 800 | mg |
| Royal Jelly Extract | 600 | mg |
| Crataegus Pinnatifida Extract | 600 | mg |
| Ziziphus Jujuba Mill. Extract | 500 | mg |
| Ginseng Extract | 220 | mg |

The amount of any individual ingredient may vary+/−10% and still maintain the same impactfulness and liver purification abilities as the preferred embodiment.

What has been described above includes examples of the claimed subject matter. It is, of course, not possible to describe every conceivable combination of components or methodologies for purposes of describing the claimed subject matter, but one of ordinary skill in the art can recognize that many further combinations and permutations of such matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

The foregoing method descriptions are provided merely as illustrative examples and are not intended to require or imply that the steps of the various embodiments must be performed in the order presented. As will be appreciated by one of skill in the art the order of steps in the foregoing embodiments may be performed in any order. Words such as “thereafter,” “then,” “next,” etc. are not intended to limit the order of the steps; these words are simply used to guide the reader through the description of the methods. Further, any reference to claim elements in the singular, for example, using the articles “a,” “an” or “the” is not to be construed as limiting the element to the singular.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

1) A liver purification supplement comprising
a) 1170-1430 mg Echinacea extract;
b) 720-880 mg pine pollen extract;
c) 540-660 mg royal jelly extract;
d) 540-660 mg Crataegus pinnatifida extract;
e) 450-550 mg Ziziphus jujuba Mill. extract; and
f) 198-242 mg Ginseng extract.

2) The liver purification supplement as in claim 1
a) wherein said Echinacea extract is present in the amount of 1200-1400 mg;
b) wherein said pine pollen extract is present in the amount of 760-840 mg;
c) wherein said royal jelly extract is present in the amount of 570-630 mg;
d) wherein said Crataegus pinnatifida extract is present in the amount of 570-630 mg;
e) wherein said Ziziphus jujuba Mill. extract is present in the amount of 475-525 mg; and
f) wherein said Ginseng extract is present in the amount of 209-231 mg.

3) The liver purification supplement as in claim 1
a) wherein said Echinacea extract is present in the amount of 1250-1350 mg;
b) wherein said pine pollen extract is present in the amount of 780-820 mg;
c) wherein said royal jelly extract is present in the amount of 590-130 mg;
d) wherein said Crataegus pinnatifida extract is present in the amount of 590-610 mg;
e) wherein said Ziziphus jujuba Mill. extract is present in the amount of 490-510 mg; and
f) wherein said Ginseng extract is present in the amount of 215-225 mg.

4) The liver purification supplement as in claim 1
a) wherein said Echinacea extract is present in the amount of 1300 mg;
b) wherein said pine pollen extract is present in the amount of 800 mg;
c) wherein said royal jelly extract is present in the amount of 600 mg;
d) wherein said Crataegus pinnatifida extract is present in the amount of 600 mg;
e) wherein said Ziziphus jujuba Mill. extract is present in the amount of 500 mg; and
f) wherein said Ginseng extract is present in the amount of 220 mg.

5) The liver purification supplement as in claim 1
a) wherein said pine pollen extract comprises a mixture of flavonoids; and
b) wherein said mixture of flavonoids comprises a first flavonoid, a second flavonoid, a third flavonoid, and a fourth flavonoid.

6) The liver purification supplement as in claim 5 wherein when said pine pollen extract is subjected to gas chromatography
a) said first flavonoid has a retention time of 3.885 minutes;
b) said second flavonoid has a retention time of 5.554 minutes;
c) said third flavonoid has a retention time of 6.023 minutes; and
d) said fourth flavonoid has a retention time of 6.740 minutes.

7) The liver purification supplement as in claim 5 wherein said mixture of flavonoids comprises
a) 37.21 percent of said first flavonoid;
b) 22.61 percent of said second flavonoid;
c) 14.94 percent of said third flavonoid; and
d) 25.24 percent of said fourth flavonoid.

8) The liver purification supplement as in claim 4
a) wherein said pine pollen extract comprises a mixture of flavonoids; and
b) wherein said mixture of flavonoids comprises a first flavonoid, a second flavonoid, a third flavonoid, and a fourth flavonoid.

9) The liver purification supplement as in claim 8 wherein when said pine pollen extract is subjected to gas chromatography
a) said first flavonoid has a retention time of 3.885 minutes;
b) said second flavonoid has a retention time of 5.554 minutes;
c) said third flavonoid has a retention time of 6.023 minutes; and
d) said fourth flavonoid has a retention time of 6.740 minutes.

10) The liver purification supplement as in claim 9 wherein said mixture of flavonoids comprises
a) 37.21 percent of said first flavonoid;
b) 22.61 percent of said second flavonoid;
c) 14.94 percent of said third flavonoid; and
d) 25.24 percent of said fourth flavonoid.

11) The liver purification supplement of claim 10 further comprising gelatine.

12) The liver purification supplement of claim 11 further comprising microcrystalline cellulose.

13) The liver purification supplement of claim 12 further comprising vegetable magnesium stearate.

14) The liver purification supplement of claim 1 further comprising gelatine.

15) The liver purification supplement of claim 14 further comprising microcrystalline cellulose.

16) The liver purification supplement of claim 15 further comprising vegetable magnesium stearate.

17) The liver purification supplement of claim 1 further comprising microcrystalline cellulose.

18) The liver purification supplement of claim 1 further comprising vegetable magnesium stearate.
